(19) United States
(12) Patent Application Publication
(10) Pub. No.: US 2007/0259913 A1
(43) Pub. Date: Nov. 8, 2007

(54) PROPHYLAXIS OF THROMBOEMBOLIC EVENTS IN CANCER PATIENTS

(76) Inventors: David Deitchman, Yardley, PA (US); Robert M. Knabb, Avondale, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/744,255

(22) Filed: May 4, 2007

Related U.S. Application Data
(60) Provisional application No. 60/797,733, filed on May 4, 2006.

(51) Int. Cl. A61K 31/4745

(52) U.S. Cl. 514/303

(57) ABSTRACT

The present invention generally relates a method of prophylaxis of thrombotic and thromboembolic events in a cancer patient in need thereof comprising administering to said patient a therapeutically effective amount of a factor Xa inhibitor, especially apixaban or a polymorph or pharmaceutically acceptable solvate form thereof. The factor Xa inhibitor may be used in combination with therapeutic agents.

This application claims the benefit of U.S. Provisional Application No. 60/797,733, filed May 4, 2006, incorporated herein by reference in its entirety.

### BACKGROUND OF THE INVENTION

Venous thromboembolism (VTE) is a common occurrence in patients with malignant disease. Compared to other groups of patients with VTE, the cancer population is unique because the pathogenesis of thrombosis differs, the frequency of VTE is greater and the clinical management required is more complex. The pathogenic mechanisms of thrombosis in the cancer patient involve a complex interaction between the tumor cell, the patient and the hemostatic system. Virchow described three classic mechanisms that play a role in thrombogenesis: stasis, activation of blood coagulation and vascular injury (Virchow R. Gesammette Abhandlungen zur Wissenchaftichen Medicin. Frankfurt, Germany: Von Meidinger Sohn, 1856: 458-636). All three are at play in patients with malignant disease. Patients with cancer are often immobile and bed-ridden as a result of their cancer or complications of cancer (e.g. infection, surgery). Also, extrinsic venous compression from tumor masses and lymphadenopathy can lead to stasis. Tumor cells can produce procoagulants (e.g. tissue factor) that activate coagulation either directly or indirectly in association with an inflammatory response. Extrinsic factors such as surgery, chemotherapy drugs and vascular access catheters can all damage the vessel wall and promote thrombogenesis.

Thrombosis has been associated with a variety of tumor types, e.g., pancreatic cancer, breast cancer, brain tumors, lung cancer, ovarian cancer, prostate cancer, gastrointestinal malignancies, Hodgkins or non-Hodgkins lymphoma, etc. Recent studies suggest that the frequency of cancer in patients with thrombosis reflects the frequency of a particular cancer type in the general population. (Levitan, N. et al. Medicine (Baltimore) 1999, 78(5):285-291; Levine M. et al. N Engl J Med 1996, 334(11):677-681; Blom, J. W. et al. JAMA: 2005, 293(6):715-722.) Hence, the most common cancers associated with thrombosis in men are prostate, colorectal, brain and lung cancer and in women are breast, ovary and lung cancer. The observed rate of VTE in cancer patients is significant. The varying rates of VTE between different tumor types are most likely related to the selection of the patient population. It is clear that risk factors for thrombosis include: the stage of the cancer (i.e. presence of metastases), the presence of central vein catheters, surgery and anticancer therapies including chemotherapy, hormones and antiangiogenic drugs.

There are three main clinical situations when considering the prevention of VTE in the medical cancer patient. The first is the patient who is bedridden for prolonged periods of time. The second involves the ambulatory patient who is receiving chemotherapy or radiation and the third involves patients with indwelling central vein catheters.

Unfractionated heparin (UFH) and low molecular weight heparin (LMWH) are effective antithrombotic agents in cancer patients undergoing surgery. (Mismetti, P. et al. British Journal of Surgery 2001, 88:913-930.) Cancer patients who are bedridden are at risk for thrombosis. Low dose UFH and LMWH have been found to be effective in patients hospitalized with acute medical illnesses. Vitamin K antagonists (VKA) such as warfarin have been used to prevent post-operative thrombosis in patients undergoing orthopedic surgery and for the secondary prevention of recurrent VTE in patients with acute VTE who have received initial treatment with UFH or LMWH.

There is relatively little data available on the primary prevention of thrombosis in ambulatory cancer patients. In one study, Levine and colleagues showed that low dose warfarin (targeted INR 1.5) was effective in reducing the rate of thrombosis in women with metastatic breast cancer receiving chemotherapy. (Levine, M. et al. Lancet 1994, 343(8902):886-889.) Despite this study, oncologists do not routinely use prophylaxis in cancer patients receiving chemotherapy with oral anticoagulants. The most likely reasons are the concern for bleeding and the logistics of laboratory monitoring and dose adjustment. The use of VKA in cancer patients can be difficult because of frequent changes in nutrition, multiple drug interactions, and alterations in liver metabolism. Central vein catheters are commonly used to deliver chemotherapy and blood products in cancer patients. Recent studies have reported rates of approximately 4-5% of symptomatic upper limb thrombosis in patients with central vein catheters. (Couban, S. et al. J Clin Oncol 2005, 23(18):4063-4069; Verso, M. et al. J Clin Oncol 2005, 23(18):4057-4062; Young, A. M. et al. J Clin Oncol (Meeting Abstracts) 2005, 23(16_suppl):LBA8004.) Two recent randomized trials, one that evaluated 1 mg of warfarin (Couban, S. et al. J Clin Oncol 2005, 23(18):4063-4069.) the other low molecular weight heparin, (Verso, M. et al. J Clin Oncol 2005, 23(18):4057-4062.) found no difference between the antithrombotic agent and placebo. However, these studies were underpowered. In a recent trial, adjusted dose warfarin at an INR of 1.5 was found to be effective in reducing central vein catheter thrombosis. (Young, A. M. et al. J Clin Oncol (Meeting Abstracts) 2005, 23(16_suppl):LBA8004.) However, it was associated with increased major bleeding.

Ambulatory medical cancer patients are at increased risk of thrombosis. Currently there is not an optimal agent to prevent thrombosis in these patients. Primary prevention of thrombosis in these patients is important because if a cancer patient develops symptomatic VTE, their clinical care becomes very complicated. They are at increased risk for recurrent thrombosis and at increased risk for anticoagulant associated bleeding. Autopsy studies have shown that a frequent cause of death in cancer patients is pulmonary embolism (PE). (Shen, V. S. et al. South Med J 1980; 73(7):841-843.) Cancer patients who develop VTE have an increased mortality rate compared to cancer patients without VTE. (Sorensen, H. T. et al. N Engl J Med 2000; 343(25):1846-1850.) Consequently, it is desirable to find new prophylactic agents for ambulatory cancer patients that are safe and effective, can be taken orally, do not require laboratory monitoring, and are well tolerated and accepted at one or more of the doses as measured by the proportion of patients remaining free of major bleeding or clinically relevant non-major bleeding.

Apixaban is a new orally active, direct inhibitor of factor Xa that binds to the active site of factor Xa without requiring antithrombin III, thus being useful for the prevention and treatment of VTE. Apixaban has the potential to fulfill an unmet medical need with a favorable benefit:risk profile in a variety of thrombotic disorders. Demonstration of a favorable benefit:risk profile in the setting of medical oncology could lead to significant reduction in serious and sometimes fatal venous thromboembolic complications of ongoing cancer and its treatment.

### SUMMARY OF THE INVENTION

The present invention provides, inter alia, a method of prophylaxis of thrombotic and thromboembolic events in a cancer patient in need thereof comprising administering to said patient a therapeutically effective amount of apixaban or a polymorph or pharmaceutically acceptable solvate form thereof.

Another aspect of the invention provides a method of prophylaxis of thrombotic and thromboembolic events in cancer patients undergoing either first or second line chemotherapy for advanced (metastatic) cancer, for example, lung cancer, breast cancer, gastrointestinal cancer (colon, rectum, pancreas, stomach), ovarian or prostate cancer; myeloma and selected lymphomas.

Another aspect of the invention provides pharmaceutical compositions comprising one or more pharmaceutically acceptable carriers or excipients and a therapeutically effective amount of apixaban or a polymorph or pharmaceutically acceptable solvate form thereof.

Another aspect of the invention provides pharmaceutical compositions further comprising at least one additional therapeutic agent.

Another aspect of the invention provides the use of apixaban or a polymorph or pharmaceutically acceptable solvate form thereof, for the manufacture of a medicament for the prophylaxis of thrombotic and thromboembolic events in a cancer patient in need thereof.

These and other features of the invention will be set forth in the expanded form as the disclosure continues.

### DETAILED DESCRIPTION OF THE INVENTION

Apixaban, disclosed in U.S. Pat. No. 6,967,208, which is herein incorporated by reference, has the chemical name 1-(4-methoxyphenyl)-7-oxo-6-[4-(2-oxo-1-piperidinyl)phenyl]-4,5,6,7-tetrahydro-1H-pyrazolo[3,4-c]pyridine-3-carboxamide and the formula

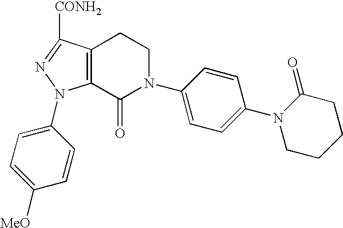

Additionally, U.S. Pat. No. 6,919,451, U.S. Patent Application Publication No. 2005/0245566 A1 and U.S. patent application Ser. No. 11/235,510 filed Sep. 26, 2005, which are herein incorporated by reference, disclose various processes and key intermediates for preparing apixaban or a polymorph or pharmaceutically acceptable solvate form thereof.

As used herein, the term “cancer patient” refers to a warm-blooded animal, such as a mammal, which is afflicted with cancer. It is understood that dogs, cats, rats, mice, and humans are examples of animals within the scope of the meaning of the term. The term “cancer” includes (but not limited to) the following: carcinoma, including (but not limited to) that of lung, breast, gastrointestinal (colon, rectum, pancreas, stomach), ovarian, uterine, prostate, bladder, thyroid, liver, kidney, head, neck and skin; tumors of the central and peripheral nervous system, including neuroblastoma, glioblastoma, and medullobalstoma; and other tumors, including melanoma, multiple myeloma, and lymphomas.

“Therapeutically effective amount” is intended to include an amount of apixaban or a polymorph or pharmaceutically acceptable solvate form thereof that is effective when administered alone or in combination to have a prophylactic effect in treating and preventing thrombotic and thromboembolic events in cancer patients.

As used herein, “prophylaxis” refers to the preventive treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

The term “pharmaceutically acceptable”, as used herein, refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

As used herein, “polymorph” refers to crystalline forms having the same chemical composition but different spatial arrangements of the molecules, and/or ions forming the crystal.

As used herein, “solvate” refers to a crystalline form of a molecule, and/or ions that further comprises molecules of a solvent or solvents incorporated into the crystalline structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate.

The term “thrombosis”, as used herein, refers to formation or presence of a thrombus (pl. thrombi); clotting within a blood vessel which may cause infarction of tissues supplied by the vessel. The term “embolism”, as used herein, refers to sudden blocking of an artery by a clot or foreign material which has been brought to its site of lodgment by the blood current. The term “thromboembolism”, as used herein, refers to obstruction of a blood vessel with thrombotic material carried by the blood stream from the site of origin to plug another vessel.

In general, a thromboembolic event or disorder is a circulatory disease caused by blood clots (i.e., diseases involving fibrin formation, platelet activation, and/or platelet aggregation). The term “thromboembolic disorders (or events)” as used herein includes arterial cardiovascular thromboembolic disorders, venous cardiovascular thromboembolic disorders, and thromboembolic disorders in the chambers of the heart. The term “thromboembolic disorders (or events)” as used herein also includes specific disorders selected from, but not limited to, unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. The medical implants or devices include, but not limited to: prosthetic valves, indwelling catheters, stents, and vessel grafts. The procedures include, but not limited to: cardiopulmonary bypass and hemodialysis. It is noted that thrombosis includes occlusion (e.g. after a bypass) and reocclusion (e.g., during or after percutaneous transluminal coronary angioplasty). The term “stroke”, as used herein, refers to embolic stroke or atherothrombotic stroke arising from occlusive thrombosis in the carotid communis, carotid interna, or intracerebral arteries.

The methods preferably comprise administering to a patient a therapeutically effective amount of apixaban or a polymorph or pharmaceutically acceptable solvate form thereof, preferably in combination with one or more pharmaceutically acceptable carriers and/or excipients. A “pharmaceutically acceptable carrier or excipient” refers to media generally accepted in the art for the delivery of biologically active agents to animals, in particular, mammals. Pharmaceutically acceptable carriers and/or excipients are formulated according to a number of factors well within the purview of those of ordinary skill in the art. These include, without limitation: the type and nature of the active agent being formulated; the subject to which the agent-containing composition is to be administered; the intended route of administration of the composition; and, the therapeutic indication being targeted. Pharmaceutically acceptable carriers and/or excipients include both aqueous and non-aqueous liquid media, as well as a variety of solid and semi-solid dosage forms. Such carriers and/or excipients can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers and/or excipients, and factors involved in their selection, are found in a variety of readily available sources such as, for example, Remington's Pharmaceutical Sciences, 17th ed., 1985, which is incorporated herein by reference in its entirety.

Apixaban or a polymorph or pharmaceutically acceptable solvate form thereof may be administered to a patient in such oral dosage forms as tablets, capsules (each of which includes immediate release, sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They may be administered alone, but generally will be administered with a pharmaceutical carrier selected on the basis of the chosen route of administration and standard pharmaceutical practice.

The dosage regimen will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder. Obviously, several unit dosage forms may be administered at about the same time. The dosage that will be most suitable for prophylaxis or treatment may vary with the form of administration, the particular crystalline form of the compound chosen and the physiological characteristics of the particular patient under treatment. Broadly, small dosages may be used initially and, if necessary, increased by small increments until the desired effect under the circumstances is reached.

Apixaban or a polymorph or pharmaceutically acceptable solvate form thereof may be administered in combination with other therapeutic agents. By “administered in combination” or “combination therapy” it is meant that apixaban or a polymorph or pharmaceutically acceptable solvate form thereof and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

If apixaban is administered in combination with therapeutic agents, the combination of compounds is preferably a synergistic combination. Synergy, as described for example by Chou and Talalay, Adv. Enzyme Regul. 1984, 22, 27-55, occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased antithrombotic effect, anti-cancer effect, improved safety profiles or some other beneficial effect of the combination compared with the individual components in the same formulation.

Additional therapeutic agents include other anti-coagulant or coagulation inhibitory agents, anti-cancer agents, anti-platelet or platelet inhibitory agents, thrombin inhibitors, thrombolytic or fibrinolytic agents, anti-arrythmic agents, anti-hypertensive agents, calcium channel blockers (L-type and T-type), cardiac glycosides, diruetics, mineralocorticoid receptor antagonists, phospodiesterase inhibitors, cholesterol/lipid lowering agents and lipid profile therapies, anti-diabetic agents, anti-depressants, anti-inflammatory agents (steroidal and non-steroidal), anti-osteoporosis agents, hormone replacement therapies, oral contraceptives, anti-obesity agents, anti-anxiety agents, anti-proliferative agents, anti-tumor agents, anti-ulcer and gastroesophageal reflux disease agents, growth hormone and/or growth hormone secretagogues, thyroid mimetics (including thyroid receptor antagonist), anti-infective agents, anti-viral agents, anti-bacterial agents, and anti-fungal agents.

Accordingly, components (a) and (b) of the present invention may be formulated together, in a single dosage unit (that is, combined together in one capsule, tablet, powder, or liquid, etc.) as a combination product. When component (a) and (b) are not formulated together in a single dosage unit, the component (a) may be administered at the same time as component (b) or in any order; for example component (a) of this invention may be administered first, followed by administration of component (b), or they may be administered in the reverse order. If component (b) contains more that one agent, these agents may be administered together or in any order. When not administered at the same time, preferably the administration of component (a) and (b) occurs less than about one hour apart. Preferably, the route of administration of component (a) and (b) is oral. Although it may be preferable that component (a) and component (b) both be administered by the same route (that is, for example, both orally) or dosage form, if desired, they may each be administered by different routes (that is, for example, one component of the combination product may be administered orally, and another component may be administered intravenously) or dosage forms.

Pharmaceutical kits which may be useful for the treatment of various disorders, and which comprise a therapeutically effective amount of a pharmaceutical composition comprising apixaban or a polymorph or pharmaceutically acceptable solvate form thereof in one or more sterile containers, are also within the ambit of the present invention. The kits may further comprise conventional pharmaceutical kit components which will be readily apparent to those skilled in the art, once armed with the present disclosure. Sterilization of the container may be carried out using conventional sterilization methodology well known to those skilled in the art.

Apixaban or a polymorph or pharmaceutically acceptable solvate form thereof may be administered orally, for example, with an inert diluent or with an edible carrier. They may be enclosed in gelatin capsules or compressed into tablets. For the purpose of oral therapeutic administration, the compounds may be incorporated with excipients and used in the form of tablets, troches, capsules, elixirs, suspensions, syrups, wafers, chewing gums and the like. For the purpose of parenteral therapeutic administration, the compounds of the present invention may be incorporated into a solution or suspension. Preferred compositions and preparations according to the present invention are prepared so that an oral dosage unit form contains between 1 and 50 milligrams of the active ingredient. More preferred compositions are prepared to contain between 2 to 20 milligrams of the active ingredient. Even more preferred compositions are prepared to contain between 5 to 20 milligrams of the active ingredient. Pharmaceutical compositions may be administered once or twice daily, preferrably once daily.

A preferred embodiment of the present invention is a Phase 2, randomized, double-blind, placebo-controlled, 4-arm trial to determine if once-daily apixaban 5 mg, 10 mg or 20 mg or matching placebo in patients receiving either first or second line chemotherapy for advanced (metastatic) cancer will be well tolerated and accepted for the prevention of thrombosis. Eligible patients must not demonstrate active bleeding or have a high risk of bleeding and patients must be enrolled within 4 weeks of starting first or second line chemotherapy. The duration of study drug treatment will be 12 weeks. The primary outcome is a composite of major bleeding or clinically relevant non-major bleeding over 12 weeks of treatment. See Table 1 for study schema.

TABLE 1
Study Design

| | | | Period X |
|---|---|---|---|
| | | | 30-day |
| | | Period C | Post-treatment |
| | Period B | 12 Week Treatment | Follow Up |
| ENROLLMENT | Screening | apixaban 5 mg QD | Telephone |
| | Randomization | or | Assessment |
| | in a | apixaban 10 mg QD | |
| | 1:1:1:1 ratio^{a} | or | |
| | | apixaban 20 mg QD | |
| | | or | |
| | | placebo | |

^{a}Stratification will be by the presence (or not) of metastatic liver disease.

Patient Inclusion Criteria
1) Patients receiving either first or second line chemotherapy for advanced (metastatic) lung, breast, gastrointestinal (colon, rectum, pancreas, stomach), ovarian or prostate cancer; myeloma and selected lymphomas (Note: lymphomas where chemotherapy is expected to cause marked thrombocytopenia, e.g. Burkitt's, are not eligible).
2) Able to begin study medication ≦4 weeks of starting either first or second line chemotherapy
3) Expected course of chemotherapy ≧90 days after start of chemotherapy
4) Men and women, aged 18 years or more

Without further elaboration the foregoing will so fully illustrate our invention that others, may, by applying current future knowledge, adopt the same for use under various conditions of service.

What is claimed is:

1. A method of prophylaxis of thrombotic and thromboembolic events in a cancer patient in need thereof comprising administering to said patient a therapeutically effective amount of apixaban or a polymorph or pharmaceutically acceptable solvate form thereof.

2. A method according to claim 1, wherein cancer patients undergoing either first or second line chemotherapy for advanced (metastatic) cancer.
